SEQUENCE LISTING (11 sequences)

SEQ ID NO: 1 - protein, 30 aa, Artificial Sequence
  Synthetic peptide for targeting mitochondria
MLSLRQSIRFFKKHKHKHKHKHKHKHKHKH

SEQ ID NO: 2 - protein, 11 aa, Artificial Sequence
  Synthetic peptide for penetrating cell
KKLFKKILKYL

SEQ ID NO: 3 - protein, 18 aa, Artificial Sequence
  Synthetic peptide for penetrating cell
LLIILRRRIRKQAHAHSK

SEQ ID NO: 4 - protein, 17 aa, Agrobacterium tumefaciens
KLRPEDRYVQTERYGRR

SEQ ID NO: 5 - protein, 17 aa, Agrobacterium tumefaciens
TLTPEDRYVQTERYGTT

SEQ ID NO: 6 - protein, 15 aa, Agrobacterium tumefaciens
KRRYGGETEIKLKSK

SEQ ID NO: 7 - protein, 15 aa, Agrobacterium tumefaciens
KTTYGGETEIKLKSK

SEQ ID NO: 8 - protein, 14 aa, Saccharomyces cerevisiae
PPAVHSFNTPAVQS

SEQ ID NO: 9 - protein, 13 aa, Saccharomyces cerevisiae
PAVHSFNTVAVQS

SEQ ID NO: 10 - DNA, 49 bp, Saccharomyces cerevisiae
caccagctgtacactcatttaatacaccagctgtacaatcttaagttat

SEQ ID NO: 11 - DNA, 94 bp, Saccharomyces cerevisiae
accagctgtacactcatttaatacagtagcgtacaatcttaagttatgatgtaactgaatgaaatggtgaaggacgggtccagtaggctgcttc

(19) United States
(12) Patent Application Publication
(10) Pub. No.: US 2020/0017865 A1
(43) Pub. Date: Jan. 16, 2020

(54) Methods for mitochondria and organelle genome editing

(71) Applicant: The Regents of the University of California, Oakland, CA (US)

(72) Inventors: Itay Budin, San Francisco, CA (US); Amanda R. Reider Apel, Alameda, CA (US); Niklas F.C. Hummel, San Francisco, CA (US); Aindrila Mukhopadhyay, Oakland, CA (US); Jay D. Keasling, Berkeley, CA (US)

(73) Assignee: The Regents of the University of California, Oakland, CA (US)

(21) Appl. No.: 16/417,499

(22) Filed: May 20, 2019

Related U.S. Application Data
(60) Provisional application No. 62/673,597, filed on May 18, 2018.

(51) Int. Cl. C12N 15/74; C12N 15/10; C12N 15/90; C12N 15/66

(52) U.S. Cl. CPC C12N 15/743; C12N 15/66; C12N 15/90; C12N 15/102

(57) ABSTRACT

The present invention provides for a method for editing a genome of a mitochondria or organelle, the method comprising: introducing a DNA fixing template into a mitochondria or organelle comprising a target DNA in the genome of the mitochondria or organelle, such that the DNA fixing template replaces the target DNA, and optionally selecting or screening for a phenotype of an eukaryotic cell comprising the mitochondria or organelle, wherein the DNA fixing template causes the phenotype.

### CROSS-REFERENCE TO RELATED APPLICATIONS

This application claims priority to U.S. Provisional Patent Application Ser. No. 62/673,597, filed on May 18, 2018, which is hereby incorporated by reference.

### STATEMENT OF GOVERNMENTAL SUPPORT

The invention was made with government support under Contract Nos. DE-AC02-05CH11231 awarded by the U.S. Department of Energy and MCB1330914 awarded by the National Science Foundation. The government has certain rights in the invention.

### FIELD OF THE INVENTION

The present invention is in the field of genome editing.

### BACKGROUND OF THE INVENTION

Mitochondria are important sub-cellular eukaryotic organelles. They are well known as organelles responsible for eukaryotic oxidative phosphorylation¹. But they also have other diversified functions, including NADH production, programmed cell death, and cell signalling²⁻⁴. Plants and algae feature an equivalent organelle responsible for photosynthesis, the chloroplast.

Due to their bacteria origin^{5-8 9,10}, mitochondria and chloroplasts are defined by presence of a distinct organelle genome¹¹. These genomes are small compared to nuclear genomes, but generally encoding essential metabolic genes, as well those required for the native translation machinery of these organelles, e.g. ribosomal RNA. For example, the Saccharomyces cerevisiae mitochondrial genome contains 35 genes, of which only 8 encode polypeptides, with the remaining genes encoding RNAs that are necessary for the translation of the polypeptides¹². These genomes often follow a modified genetic code. For example, the S. cerevisiae mitochondrial genome contains an extra methionine codon, and one less termination codon. Additionally, 4 of the leucine codons (CUN) are threonine codons in the mitochondria. The copy number of organelle genomes is quite high—10s to 1000s—and copies of the mitochondrial genome are collected together into nucleoids¹³.

Transcription and translation also differ in these organelles, due to their proteobacterial origins. In S. cerevisiae mitochondria, Rpo41p is the RNA polymerase responsible for all of the transcription¹⁴ and potentially plays a role in the replication of the mitochondrial genome¹⁵. Unlike the bacteriophage RNA polymerases that it is closely related to, it requires an additional factor for stable promoter binding, Mtf1p^{16,17}. As for most of the proteins in the mitochondria, the genes of both of these are found in the nuclear genome and the polypeptides are imported into the mitochondria first through an outer membrane translocase (TOM), and then an inner membrane translocase (TIM). For proteins localized to the mitochondrial matrix, there is a cleavable N-terminus localization tag. These tags (presequences) guide the proteins through the outer and inner membrane translocase complexes¹⁸. Once the protein enters the matrix, the presequence is cleaved by MPP (mitochondrial processing peptidase). Chaperones then help the protein to re-fold correctly¹⁹.

Since the development of CRISPR/Cas9, there has been much activity to develop tools for the cheap and quick genomic engineering of many different host organisms²⁶⁻²⁴. Less work has been focused on how it can be applied to the engineering of the mitochondrial genome, but Jo et al., (2015) proved some evidence that expression of Cas9 could induce toxicity in human embryonic kidney cells. All endonuclease-mediated genome editing relies on native or introduced homologous repair machinery to introduce genome edits induced or selected for by site-specific cutting of the genome locus. Double-strand break (DSB) repair in mitochondria can be mediated both by homologous recombination (HR), and non-homologous end-joining (NHEJ) ²⁶⁻²⁹. Due to the high number of copies of medina, these pathways may not be as highly developed as those for nuclear DSBs³⁰.

S. cerevisiae is a valuable model to study mitochondrial editing because of its ability to survive without functional mitochondria³¹. It is also possible to transform mitochondrial DNA (medina) in this system via micro particle bombardment (biolistic). This method involves the coating of microparticles (e.g. 0.6 μm diameter gold particles) with DNA, which are fired at high velocity into the yeast cells. Established methods rely on cells that either lack the entire medina) (rho⁰), part of the medina (rho⁻), or still contain the full medina (rho+). rhO⁰ strains can be transformed with synthetic rho⁻ molecules. These strains can then be mated with a rho⁺ strain, whereby the process of homologous recombination will introduce the synthetic rho⁻ fragment into the medina. It is possible to obtain rho⁺ final strain without the mating step, if the introduced alteration can be selected for^{31,32} This process is cumbersome and limited, and therefore has been comparatively little genetic work on modifying the mitochondrial DNA.

### SUMMARY OF THE INVENTION

The present invention provides for a method for editing a genome of a mitochondria or organelle, the method comprising: introducing a DNA fixing template into a mitochondria or organelle comprising a target DNA in the genome of the mitochondria or organelle, such that the DNA fixing template replaces the target DNA, and optionally selecting or screening for a phenotype of an eukaryotic cell comprising the mitochondria or organelle, wherein the DNA fixing template causes the phenotype.

The present invention provides for a method for editing a genome of a mitochondria or organelle, the method comprising: (a) introducing a targeted endonuclease, a targeting guide RNA, and a DNA fixing template into a mitochondria or organelle comprising a target DNA in the genome of the mitochondria or organelle, such that the DNA fixing template replaces the target DNA, and (b) optionally selecting or screening for a phenotype of an eukaryotic cell comprising the mitochondria or organelle, wherein the DNA fixing template causes the phenotype.

This present invention provides for a method for making homogenous sequence modifications (edits or integration) into the mitochondrial genome of eukaryotic cells. Previous gene editing tools have focused on the nuclear genome, which encodes for the vast majority of cellular genes. However, mitochondrial DNA (medina) encodes several essential molecular components involved in respiration, and therefore could be targets of metabolic engineering. Mutations in medina are heavily implicated in human mitochondrial diseases, and repair of these in somatic or germline cells is a potential avenue for novel therapeutics. Unlike the nuclear genome, medina exists in high copy numbers in a single cell, thereby serving as an attractive target for high-level, heterologous gene expression in biotechnology hosts, such as yeast. However, this same feature presents a challenge for introducing homogenous genetic changes, which this invention provides a method for overcoming.

Two further novel methods for delivering DNA (such as substrates for recombination-based gene editing) into the mitochondria of eukaryotic cells are described as follows:

The present invention provides for a method for editing a genome of a mitochondria or organelle, the method comprising: (a) introducing a peptide-DNA complex comprising a peptide that targets a mitochondria complexed with a DNA fixing template into a mitochondria or organelle comprising a target DNA in the genome of the mitochondria or organelle, such that the DNA fixing template replaces the target DNA, and (b) optionally selecting or screening for a phenotype of an eukaryotic cell comprising the mitochondria or organelle, wherein the DNA fixing template causes the phenotype.

In some embodiments, one or more peptide-DNA complexes that deliver DNA into the mitochondria. Novel peptide sequences that target the mitochondria are designed and tested. When complexed to DNA of arbitrary sequences, these peptides cause its incorporation into the mitochondrial matrix. In a particular embodiment, DNA delivery is assayed by transient expression of a mitochondrial-coded reported gene, such as a GFP gene.

The present invention provides for a method for editing a genome of a mitochondria or organelle, the method comprising: (a) providing one or more modified Agrobacterium tumefaciens Vir proteins wherein the one or more modified A. tumefaciens Vir proteins that target a DNA fixing template flanked by T-DNA recognition sequences native to the Vir system into a mitochondria or organelle instead of a nucleus, (b) introducing the one or more modified A. tumefaciens Vir proteins complexed to the DNA fixing template into an eukaryotic cell, (c) delivering the DNA fixing template into a mitochondria or organelle of the eukaryotic cell such that the DNA fixing template replaces a target DNA in the mitochondria or organelle, and (d) optionally selecting or screening for a phenotype of an eukaryotic cell comprising the mitochondria or organelle, wherein the DNA fixing template causes the phenotype.

In some embodiments, the Agrobacterium tumefaciens virulence machinery for T-DNA targeting is hijacked. Rational engineering of virulence (Vir) genes from Agrobacterium tumefaciens is used to target them away from the nucleus and into the mitochondria, which is confirmed by microscopy of fluorescent protein fusions. These proteins then deliver designer DNA into mitochondria provided that it is flanked by T-DNA recognition sequences native to the Vir system.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1: Graphical schematic. Technologies are presented for the introduction of targeted endonucleases (blue) and DNA fixing templates (red) into eukaryotic organelles. Endonuclease activity acts as a selection for the incorporation of the fixing template, allowing for homoplasmic changes in organelle composition. Homogenous modifications of mitochondrial genetics. A mitochondrial imported Cas9 (mtCas9, blue) complexed with a single guide RNA (sgRNA, purple) targets a medina locus for cutting. An imported fixing template (red) is introduced, repairing cut medina copies. Un-fixed medina copies are under continual negative selection by mtCas9-cutting, thereby leading to homogenous editing of the mitochondria.

FIG. 2: Import of Cas9 into the mitochondria. A) Because of an introduced mitCas9 becomes entirely localized within yeast mitochondria, which are labelled with a peripheral mitochondrial protein, Fis1. B) Mitochondrial imported Cas9 co-localizes with mitochondrial nucleoides, labelled with Abf1, indicating its association with mitochondrial DNA.

FIG. 3: Methods for importing RNA into the mitochondria. A) Nuclear expression of a sgRNA that tightly binds a mitochondrial-targeted Cas9 allows for its import via TIM/TOM channels through the Cas9 MTS. B) Expression of a fusion RNA containing both a single guide RNA and a motif from tRK1 allows for import via the cell's native Lysine tRNA import pathway.

FIG. 4: Quantifying sgRNA import into the mitochondria. A) Quantitative reverse transcriptase PCR (qRT-PCR) is used to detect sgRNA in purified mitochondria. Shown are raw amplification plots. A mitochondrial transcript, COX2, is also detected in the sample, but a nuclear transcript, ACT1, is not, indicating a purified sample free of nuclear RNA contaminants. B) The sgRNA abundance in the mitochondria is enhanced by co-expression of Cas9, which can bind it tightly and carry it through the mitochondrial import channels. In the absence of mtCas9 (W303a), only trace amounts of guide RNA are detected.

FIG. 5: Imported Cas9 and sgRNA act to target a mitochondrial gene. Expression of COX1 is assayed by qRT-PCR. (left) Only with the presence of a mitochondrial Cas9 (YAI5) does expression of a COX1 sgRNA allow for significant (˜100-fold) reduction of COX1 expression. (right) Only in the presence of a COX1 sgRNA does a mitochondrial Cas9 lead to a reduction in COX1 expression.

FIG. 6A: Targeting an essential gene selects against unedited locus. Method for screening for mitochondrial loss by transformation followed by testing growth on a non-fermentable carbon source (SEG-URA) that requires functional mitochondria.

FIG. 6B: Targeting an essential gene selects against unedited locus. Mitochondrial Cas9 in conjunction with a COX1 sgRNA causes significant loss in mitochondria. This effect is increased with higher sgRNA expression (2μ ori vs. CEN plasmid).

FIG. 6C: Targeting an essential gene selects against unedited locus. Mitochondrial loss due to COX1 targeting is dependent on the expression level of mitochondrial Cas9. Cas9 expression is modulated with a constitutive promoter library based on the TEF1 promoter, and is quantified by fluorescence of a Cas9-GFP construct using flow cytometry.

FIG. 7A: Integration of a fluorescent Spinach aptamer at the COX1 locus. Strategy for integration. A Cas9/sgRNA targets a PAM sequence on the c-terminus of the COX1 gene. The DNA fixing template, introduced via biolistic bombardment, mutates the PAM sequence and introduces a Spinach2 aptamer sequence on its 3′ untranslated region. An imported mtCas9, an sgRNA targeting the COX/locus, and a DNA fixing template are used to tag the 3′ UTR of COX1 mRNA with a fluorescent aptamer (Spinach2).

FIG. 7B: Integration of a fluorescent Spinach aptamer at the COX1 locus. Spinach2 integration leads to increased fluorescence, as measured with flow cytometry when incubated with the Spinach ligand (DFHBI). This effect can be used to screen individual biolistic transformants for medina integration.

FIG. 7C: Integration of a fluorescent Spinach aptamer at the COX1 locus. Fluorescence micrographs showing the background strain (i), a nuclear Spinach2 integration that serves as positive control (ii), and a mitochondrial Spinach2 integration, after incubation with the ligand. Scale bar, 20 μm. The amino acid sequence of COX is SEQ ID NOs: 8 and 9. The corresponding nucleotide sequence is SEQ ID NOs: 10 and 11.

FIG. 8: Model for re-engineering the native Agrobacterium VirD1/VirD2 system (top) for DNA transformation into the mitochondria (bottom).

FIG. 9A: Peptide-DNA complexes that deliver DNA into the mitochondria. Schematic: a complex of two synthetic peptides (mitochondrial targeting peptide (MTP) and a cell penetrating peptide (CPP)) with the delivered DNA (pDNA) is transformed into cells, where it is targeted to the mitochondria where the pDNA is released.

FIG. 9B: Peptide-DNA complexes that deliver DNA into the mitochondria. Peptide-DNA complex formation assayed by electromobility retardation of pDNA with increasing amounts of peptide.

FIG. 9C: Peptide-DNA complexes that deliver DNA into the mitochondria. Design of a short (tOxaIV) mitochondrial targeting sequence (MTS) that targets GFP-tagged complexes to the mitochondria (imaged by the mitochondrial marker Fis1-mCherry).

FIG. 9D: Peptide-DNA complexes that deliver DNA into the mitochondria. Demonstration of DNA delivery by transient expression of an mtDNA-encoded GFP gene off of the delivery pDNA. GFP is detected with increased bulk fluorescence (top) over the pDNA or peptide-free controls (green vs. black, red) and localized fluorescence in the mitochondria (bottom).

FIG. 10A: Hijacking the Agrobacterium tumefaciens virulence machinery for DNA targeting to mitochondria. Schematic of the approach in which key proteins VirD2 and VirE2 are engineered so they not delivered to the nucleus but instead carry T-DNA encoded sequences to the mitochondria (mtDNA).

FIG. 10B: Hijacking the Agrobacterium tumefaciens virulence machinery for DNA targeting to mitochondria. Rational mutation of VirE2 nuclear localization sequences (NLS1 and NLS2) from their native, nucleus-targeted state (top) to the engineered versions used here (bottom). The sequence of NLS1 is SEQ ID NO:4, and the sequence of the mutant NLS1 is SEQ ID NO:5. The sequence of NLS2 is SEQ ID NO:6, and the sequence of the mutant NLS2 is SEQ ID NO:7.

FIG. 10C: Hijacking the Agrobacterium tumefaciens virulence machinery for DNA targeting to mitochondria. Rational engineering of virulence (Vir) genes from Agrobacterium tumefaciens was used to target them away from the nucleus and into the mitochondria, which was confirmed by microscopy of fluorescent protein fusions to native VirE2 (top), which localizes to the nuclear chromosomes, and the engineered version (bottom), which co-localizes to mtDNA nucleoids labeled by Abf2-mCherry.

FIG. 10D: Hijacking the Agrobacterium tumefaciens virulence machinery for DNA targeting to mitochondria. Similar engineering of VirD2 relocalizes it from the nucleus (top) to mitochondria (bottom). These proteins then deliver designer DNA into mitochondria provided that it is flanked by T-DNA recognition sequences native to the Vir system.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)), which is incorporated herein by reference. The nomenclatures utilized in connection with, and the laboratory procedures and techniques described herein are those well known and commonly used in the art.

These and other objects, advantages, and features of the invention will become apparent to those persons skilled in the art upon reading the details of the invention as more fully described below.

In some embodiments, the targeted endonuclease is Cas9. In some embodiments, the DNA fixing template comprises a nucleotide sequence that is heterologous to the mitochondria or organelle.

Any eukaryotic cell can be used in the present method. Suitable eukaryotic cells include, but are not limited to, fungal, plant, insect or mammalian cells. Suitable fungal cells are yeast cells, such as yeast cells of the Saccharomyces genus, such as Saccharomyces cerevisiae. In some embodiments, the organelle is a chloroplast. In some embodiments, the cell is a plant cell, and the organelle is a chloroplast.

A targeted endonuclease (Cas9, a RNA-guided DNA endonuclease) is modified for import into the mitochondria. In conjunction with novel methods for nucleic acid delivery, this system allows for homoplasmic (found in all DNA copies) changes in the mitochondrial genome. Previous applications of similar gene editing technology have focused on the nuclear genome, which has distinct import pathways and generally exists in haploid (single chromosomal copies of each chromosome) or diploid (two copies of each chromosome). In contrast, the mitochondrial genome exists in many copies (such as hundreds of copies) per cell (polyploidy). This feature is beneficial for proposed applications (e.g. high expression of metabolic pathways), but makes homoplasmic edits difficult to achieve in the absence of a selection against un-edited copies. By using an endonuclease that specifically targets un-edited DNA loci, one can achieve efficient, homoplasmic integrations in the mitochondrial genome. Integrations include changes to native mitochondrial genes to improve and modify host metabolism, and the introduction of heterologous metabolic pathways for bioproduction. This present invention also provides for the following: develop novel protein-based methods for the import of accessory RNA and DNA that are needed for genetic modifications. These methods are novel and are distinct from previously developed methods for import into the nucleus currently used for nuclear DNA editing.

This invention serves as a broad-range technology platform for making modifications to mitochondrial genomes. It is useful the following: (a) modify the medina-encoded central metabolic machinery in industrial biotechnology host cells, (b) introduce highly expressed heterologous metabolic pathways into industrial biotechnology host cells, and/or (c) repair DNA mutations in the mitochondria of human cells responsible for disease.

Existing technologies for modifying medina require a selection, generally as complementation of a mutated gene, to achieve homoplasmic integration. This technology allows for any genetic change of the medina without a selection marker or other sequence constraints. The invention also provides novel techniques for importing of nucleic acids (DNA and RNA) needed for Cas9-based gene editing into the mitochondrial matrix. This invention therefore allows for efficient, reliable genetic engineering in the mitochondria, which has not been previously possible.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Mitochondrial and Organelle Genome Editing

Genome editing has fast become an indispensable tool in modern metabolic engineering, and synthetic biology applications. The relative low cost and ease of use has sped up the rate at which scientists can manipulate their organisms of interest. The use of targeted and programmable endonucleases, such as bacterial Cas9, are now established in eukaryotic systems, but so far has only been applied to nuclear genetic engineering. Organelle genomes open up new possibilities and present challenges for genetic engineering because of their essential roles in metabolism and their high copy numbers. To this end, the methods are introduced: (1) inducing and selecting for changes in the mitochondrial genomes by importing endonucleases and associated targeting guide RNA into the mitochondria, (2) selecting and screening for the introduction of heterologous DNA into the mitochondrial genome, and/or (3) introducing DNA fixing templates using genetic and chemical means. These methods will assist in metabolic engineering efforts for products derived from TCA cycle intermediates and other mitochondrial-derived metabolites in yeast, and provide technology for organelle editing in other systems, e.g. mitochondria in human cells and chloroplasts in plants/algae.

### Materials and Methods

### Strains and Plasmids

Strain W303-1A is used as the parental S. cerevisiae strain in this study. For plasmid storage and propagation, Escherichia coli DH5a is used. A complete list of strains can be found in the supplementary materials. Plasmids are constructed according to the Gibson method³³.

### Media and Growth Conditions

E. coli are either grown in liquid Luria-Bertani media with 100 μg mL⁻¹ carbenicillin, or on LB agar plates containing 75 μg mL⁻¹ carbenicillin.

For general purposes, complete supplement mixture (CSM) amino acid drop out agar plates are used for yeast propagation (VWR International). In the case of the biolistic transformations, fresh plates are made on the day of the experiment; 136.6 g L⁻¹ mannitol, 136.6 g L⁻¹ sorbitol, 0.77 g L⁻¹-Ura drop-out powder, 25 g L⁻¹ granulated agar, 6.7 g L⁻¹ yeast nitrogen base without amino acids, and after autoclavation 100 ml of sterile 20% glucose solution added.

Yeast propagation in liquid media is either carried out in YPD (20 g L⁻¹Bacto peptone, 10 g L⁻¹ yeast extract, 20 g L⁻¹ dextrose), YPEG (instead of dextrose; 30 ml glycerol, 30 ml ethanol), or CSM (0.77 g L⁻¹ CSM dropout powder, 6.7 g L⁻¹ yeast nitrogen base without amino acids, 20 g L⁻¹ dextrose).

### Chemicals and Reagents

Unless specified, all chemicals and reagents are purchased from Sigma-Aldrich.

### Flow Cytometry

Cells transformed with the Spinach aptamer are inoculated into either YPD, or YPEG in 96 deep well plates overnight at 30° C. with shaking at 250 rpm. After sufficient growth is observed, the ligand (what), is added to a final concentration of 100 μm and incubated for 1 hr at 30° C. Samples are arranged in a 96-well format. Each sample is automatically shaken for 10 seconds, prior to 10,000 events per well being analyzed and fluorescence read at 533/30 nm, with excitation at 488 nm. Flow cytometry experiments are performed on a BD Accuri C6 (BD Biosciences, US).

### Fluorescence Microscopy

Cells are immobilized onto a slide with concanavalin A (MP Biomedicals), and performed on a Zeiss LSM 710 Confocal Laser Scanning Microscope (Carl Zeiss AG). Excitation for GFP is set to 488 nm, and emission is recorded at 505 nm. Excitation for mCherry is set to 587 nm, and emission recorded at 610 nm.

### Sequencing

For plasmid verification, Sanger sequencing is performed by Genewiz (United States). For sequencing of the mitochondrial genome, the DIVA sequencing service at the Joint BioEnergy Institute is used.

### Transformations

For transformations of plasmids, or genes into the nuclear genome, the protocol of Gietz and Schiestl (2007) is followed. After transformation, the strains are plated onto either CSM agar plates, or for transformations with the KanMX cassette, onto YPD agar plates containing 200 mg L⁻¹ G418.

For the biolistic transformations, strain W303a-mtCas9 is grown overnight in 5 ml YPD, 30° C. at 250 rpm shaking in 14 ml culture tubes. The following morning, this is diluted 1:10 in fresh YPD (25 ml) in baffled shake flasks. After 3-4 hrs of growth, freshly prepared osmotic transformation plates (-Leu) are coated with 1 ml of 2 OD₆₀₀. During this time, gold microcarrier particles (1 μm diameter) are coated with 1 μg of DNA per transformation. The Biolistic® PDS-1000/He Particle Delivery System (Bio-Rad) is used for the bombardments, with parameters used as outlined in the product manual. A vacuum pressure of 28 inches Hg, and 1,350 psi rupture disks are used.

### Mitochondrial Isolation and RNA Isolation

Following the manufacturers protocol for the Mitochondrial Yeast Isolation Kit, centrifugal sub-cellular fractionation isolated the mitochondria. The YeaStar™ RNA kit isolated mitochondrial RNA, following the manufacturer's protocol. The Ambion® TURBO DNAfree™ kit (Life Technologies, Corp.) removed possible DNA contamination.

### Expression Analysis and RNA Abundance Measurement

The purified mitochondrial RNA samples are tested for their levels of sgRNA through qRT-PCR, using the Invitrogen SuperScript® III Platinum® SYBR® Green One Step qRT PCR kit (Life Technologies, Corp.). Act1 amplification provided a negative control to test for nuclear genomic contamination. Cox2 served as the reference gene to calculate RNA concentration. A series of 10 dilutions, between 100 fg to 100 ng, determined the cycle threshold (CT) value for each dilution, producing the calibration curve.

### Petite Colony Formation

Both the WT strain without mitCas9 expression, and strain YAIS with mitCas9 expression are transformed with plasmids containing sgRNA expression cassettes. These sgRNA cassettes are designed to target the mitochondrial gene COX1. Two versions are tested, one being expressed from a low-copy number plasmid (CEN ori), and the other from a high-copy number plasmid (2μ ori). The negative control consisted of a sgRNA cassette targeting the nucleus. After transformants appeared, each plate is counted for the numbers of normal sized colonies vs petite colonies. For each transformation, 3-4 plates are counted.

### Results

### Localization of an Endonuclease to the Mitochondrial Matrix

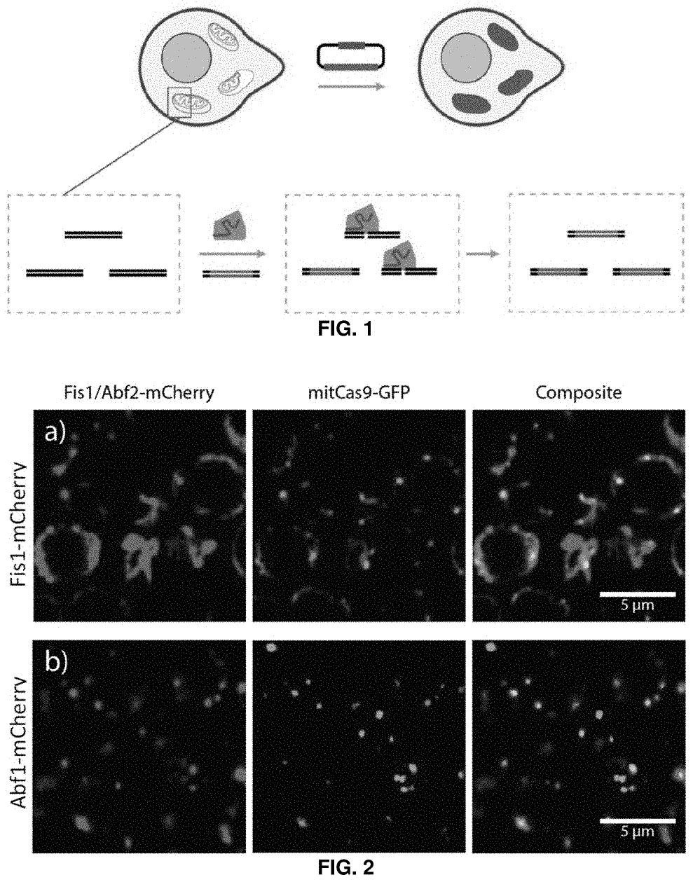

It is first sought to target a programmable endonuclease, in this case Cas9 from S. pyogenes, into the mitochondria of S. cerevisiae. For this, different N-terminal mitochondrial targeting sequences (MTS) are screened for their ability to localized a C-terminal GFP tagged Cas9 into yeast mitochondrial. This would allow mitochondrial-targeted Cas9 (mitCas9 or mtCas9) to enter into the mitochondria through protein uptake channels (TIM/TOM complex). FIG. 1 shows data for a nuclear-expressed mtCas9-GFP featuring an MTS from ATP Synthase subunit 9 from Neurospora cassa. Similar data has been obtained with other MTS from yeast species, e.g. COX4 from S. cerevisiae. This strain is transformed with two different plasmids to assay mtCas9 localization. The first, expresses a FIS1-mCherry construct; Fis1p is a peripheral mitochondrial protein that binds to the outer membrane, thereby staining the organelle. The second, contains an ABF2-mCherry construct; Abf2 is a mitochondrial nucleoid protein that forms puncta where nucleoids are present. The green signal from mtCas9-GFP lies within the red signal from Abf2-mCherry, indicating that the Cas9 is being successfully located to the mitochondria (FIG. 1). Further, mtCas9 completely co-localizes with Abf2-mCherry cells, indicating that Cas9 is imported into the mitochondria and associating with medina. This is consistent with the affinity of Cas9 to associate with dsDNA, even in the absence of a gRNA^{24,35}.

Methods for RNA Import into the Mitochondria

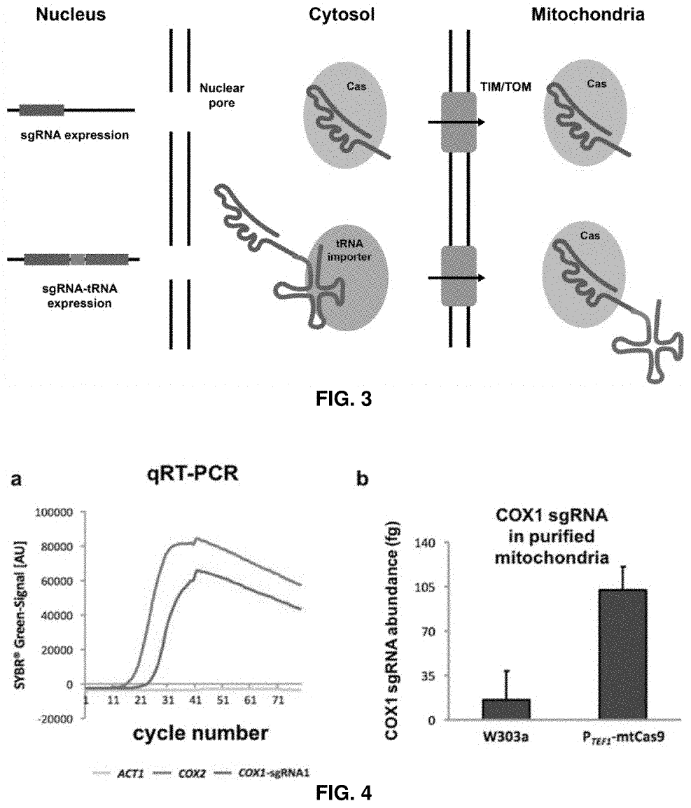

Targeted endonucleases based on CRISPR systems require RNA molecules (guide RNAs) to target to a specified locus. Cas9 can function with just a single guide RNA (sgRNA) that is a fusion molecule of tracrRNA and crRNA molecules (Jinek et al., 2012). However, there are no nucleic acid importers for the mitochondria, as rRNAs and tRNAs are generally encoded on the mitochondrial genome. Two mechanisms for RNA import are presented. The first is to co-import the along with a tightly bound protein, e.g. Cas9, through the protein import channel (TIM/TOM) (Method 1). The second is as a fusion molecule with a tRNA that is imported by the cell into the mitochondria (Method 2). Although most tRNAs used for mitochondrial translation are encoded in the mitochondrial genome, a small fraction are imported after nuclear transcription and processing. In yeast, for example, there are three lysine tRNAs³⁶, one of which ((Lys)CUU) is encoded by the nuclear gene tRK1, but present both in the cytosol and mitochondria. The import of this molecule is dependent of binding to a protein (enolase) to the 3′ hydroxyl and stem loops of the tRNA, which allows for its transport through the mitochondrial membrane through the TIM/TOM machinery³⁷′³⁸. By expressing a fusion construct in which the sgRNA is affixed to 5′ end of tRK1, the sgRNA can be co-imported into the mitochondrial matrix with the tRNA analogue. This method can function in addition to Method 1, both of which are summarized in FIG. 3.

To test sgRNA import into the mitochondria, qRT-PCR on RNA is isolated from mitochondria (FIG. 4). For controls, the mRNAs for ACT1, a nuclear transcript that should be absent in the mitochondria, and COX2, a mitochondrial transcript that should be plentiful in the mitochondrial, are also monitored. A plasmid-expressed sgRNA targeting COX1 is robustly detected by qPCR (FIG. 4) but only in the presence of a mitochondrial targeted Cas9. These experiments showed that co-expression of a mitochondrial Cas9 allowed for robust import of a COX1 sgRNA. In the absence of Cas9-expression (W303a, base strain), only trace amounts of COX1 sgRNA signal is detected, indicating it is not robustly imported. These findings support Method 1 for guide RNA import.

To test the function of mitochondrial imported Cas9 and a medina-targeting sgRNA, expression of COX1 is assayed, as targeting of the locus would result in aborted transcripts. qRT-PCR is run at locus surrounding the cut site in strain backgrounds with (YA5) or without (W303a) mitCas9 and with (pCOX1sgRNA) or without (EV) plasmids expressing the COX1-targeting sgRNA. These experiments showed a ˜100-fold decrease in COX1 expression levels with both Cas9 and the COX1 sgRNA, demonstrating efficient and specific mitochondrial gene disruption.

### Disruption of Essential Medina Genes as a Selection for Homoplasmic Gene Editing

The challenge in mitochondrial genome editing is achieving homoplasmic (in all copies) modifications, which requires a selection against un-edited genome copies. To achieve this, a mitochondrial endonuclease (e.g. mitochondrial tCas9) is targeted to target a site (a PAM site for Cas9) on an essential gene that is required for respiration, such as COX1. The DNA sequence to be integrated then includes a silent or neutral mutation at this target site, thereby protecting edited copies from endonuclease targeting. Selection against un-edited genome copies is most effective under conditions in which optimal mitochondrial function is required, such as growth under non-fermentable carbon sources for yeast.

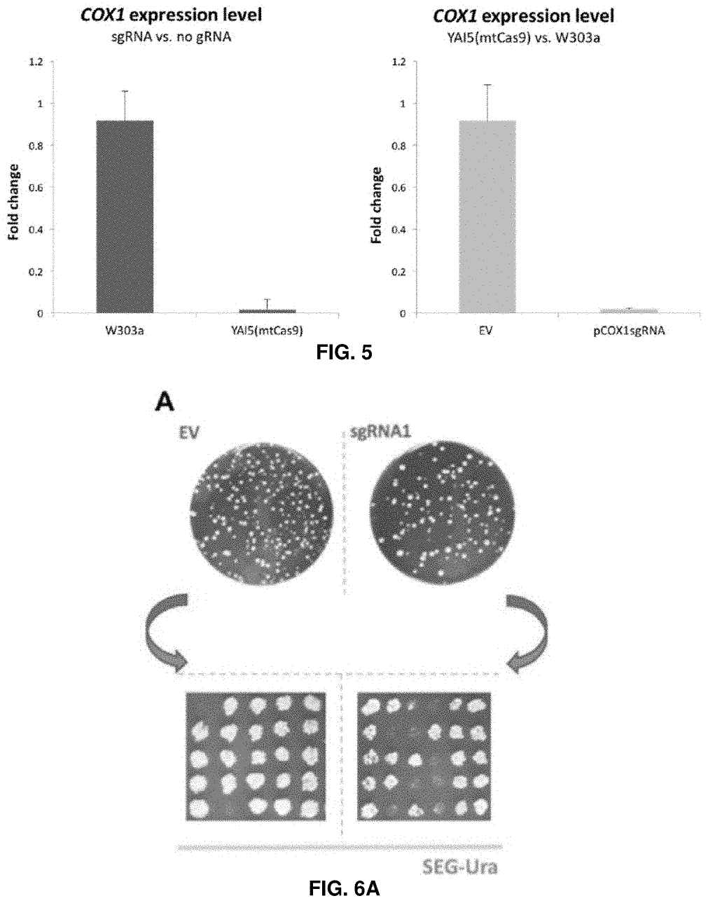

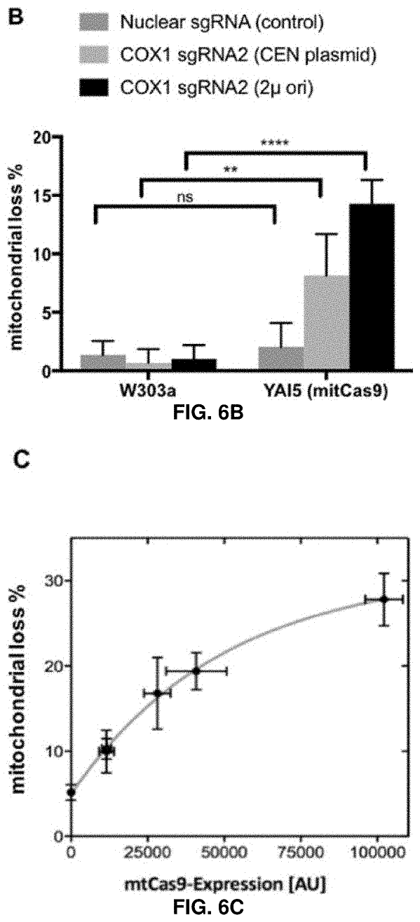

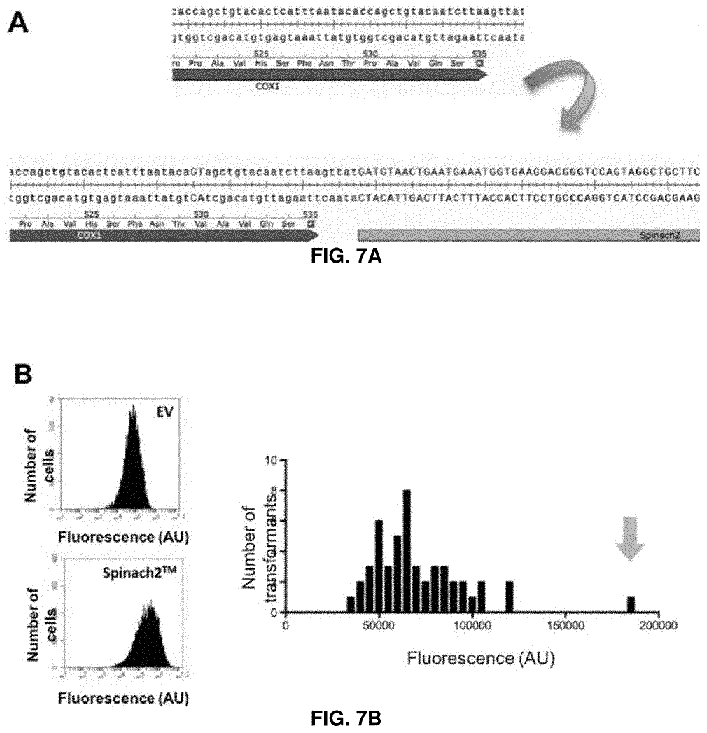

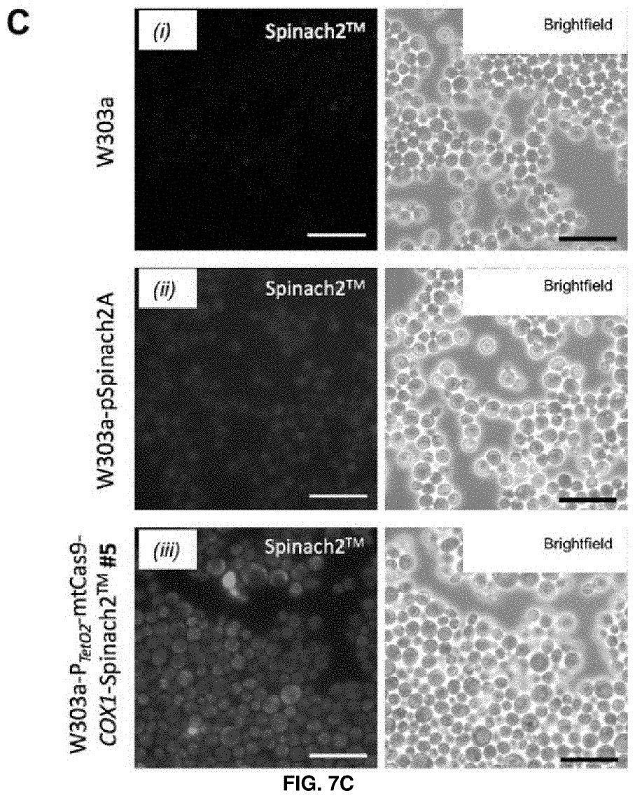

To demonstrate this principle, the effects of a mitCas9 in strain YAI5 coupled with expression of a sgRNA targeting COX1 is assayed (FIG. 5). Cells either expressing the endonuclease (YAI5) or not (W303) are transformed with plasmids expressing the COX1 sgRNA or, as a control, one targeting an essential nuclear gene. After transformation, individual colonies are screened by their ability to grow on a non-fermentable carbon source media (SEG)³⁹, allowing quantification of the fraction of cells that lost mitochondrial function (FIG. 5). Only cells expressing both mitochondrial Cas9 and the mitochondrial sgRNA showed a significant increase in mitochondrial loss (FIG. 5). This selection is increased with higher expression of the sgRNA using a high copy plasmid (2μ vs. CEN, a low copy plasmid) and with increasing mitCas9 expression, which is assayed by changing its promoter strength (FIG. 5).

### Methods to Screen for Positive Integrants

### 1. Screening Via Co-Integration of Fluorescent RNA Reporters

To test for and screen integrations into the mitochondrial genome, a DNA sequence encoding a Spinach RNA aptamer (Spinach2) is inserted. Spinach2 fluoresces when bound to an externally-supplied ligand (DFHBI), acting as a mimic of GFP⁴⁰. To aid in integration, mtCas9 is used in conjunction with a previously constructed gRNA that recognizes a 20 bp sequence at the 3′ end of COX1 gene. Disruption of this gene leads mitochondrial loss, and as such can be used as a selection. At this cut site a fixing template that includes homology surrounding the cut site is inserted, the targeted PAM site is mutated, and includes an adjacent Spinach2 RNA aptamer that is co-expressed in the 5′ UTR. The fixing template for this insertion is introduced via biolistic transformation. By using a fluorescent reporter, transformants are rapidly screened for their fluorescent expression with a flow cytometer. Shown in FIG. 4 is a representative histogram of a control population against one with the spinach aptamer inserted. This provides evidence that one is able to insert a section of DNA into a predefined locus using the mitochondrial targeted Cas9 and a gRNA targeting the 3′ an essential gene. Additionally, this strategy allows for addition of adjacent DNA sequences (e.g. gene insertions) whose incorporation would be screened for by the Spinach2 fluorescence.

### 2. Screening Via Co-Induction of Antibiotic Resistance

Erythromycin is a macrolide antibiotic which can act as a bactericide or bacteriostatic agent against many types of bacteria⁴¹. It generally acts on bacteria, binding the 505 ribosomal subunits to inhibit protein synthesis. However, It also is effective against mitochondrial ribosomes⁴². Two SNPs have been previously identified to confer erythromycin resistance in yeast mitochondria^{43,44}, both in the 21S rRNA. The first is an A to G substitution at bp 1951 of 21S rRNA, and the other is C to T at bp 4005. In addition, by genome screening spontaneous mutants, it is observed that A to T at bp 1951, as opposed to A to G in Sor and Fukuhara, (1982), also induced resistance. These mutations likely confer resistance by altering the conformational structure of the 21S rRNA.

Erythromycin can be used as a selection or screening agent for medina integrants in conjunction with the mtCas9 system described above. In our system, the fixing template introduced a desired gene construct adjacent to the 21S rRNA, while also mutating the 21S sequence to induce erythromycin resistance. This strategy can be used in conjunction with a mitochondria targeted endonuclease, as described above.

Novel Mechanism of DNA Delivery into the Mitochondria Using Protein Conjugates

The above integrations are achieved by incorporating a synthetic DNA fixing template that is introduced via biolistic bombardment, in which DNA is absorbed onto gold nanoparticles that are shot at the cell sample and, with low frequency, enter the mitochondria or other organelles. This method has many limitations, e.g. low efficiency, lack of scalability, and limitation to a small number of organisms that can withstand the bombardment.

A novel means of introducing DNA into the mitochondria or other organelles using proteins that covalently bind to double strand or single strand DNA is introduced. MTS or chloroplast targeting sequences are added to these proteins, constructing novel fusions, which are then targeted to the organelle. Because of the covalent linkage, these fusion proteins carry their bound DNA with them across the import channels.

This concept is tested with VirD2, and its accessory protein VirD1, from Agrobacterium tumefaciens⁴⁵⁻⁴⁷. In the native system, the Agrobacterium proteins VirD1 and VirD2 recognize border 25 bp on a virulence plasmid, nicking and unwinding the bottom strand they border (T-strand) which generally contains virulence (vir) genes. After release, VirD2 remains associated with the 5′ end of the T-strand through a covalent attachment to a conserved Tyrosine residue. The VirD2/T-strand complex is then injected into the bacteria's eukaryotic target (host) cell, where a C-terminal nuclear localization sequence (NLS) on the VirD2 guides it to the nucleus for integration into the host genome.

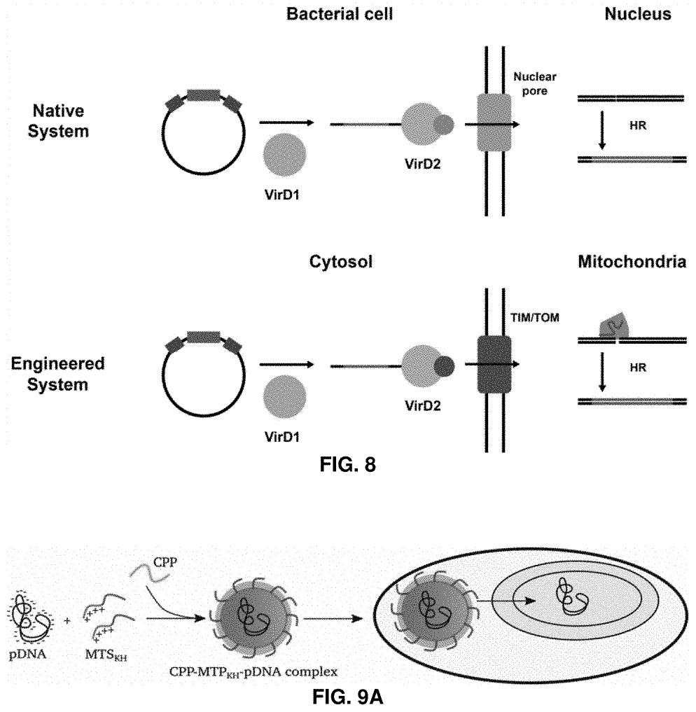

In our engineered system, VirD1 and VirD2 act to release a sequence or gene of interest to be integrated into the mitochondrial genome that contains homology to a site on the mitochondrial DNA locus. A new version of VirD2 is engineered so that the NLS is removed and an N-terminal MTS is introduced. Because VirD2 remains covalently bound to the T-strand, it carries the DNA across the TIM/TOM complex due to the ability of peptide-DNA conjugates to co-transport into mitochondna^{48,49}. This system is described in FIG. 8.

The engineered VirD1/VirD2/T-strand system can be introduced in one of three ways to cell's cytoplasm: 1) VirD2-T DNA is prepared in vitro⁴⁷ using purified proteins. The complex is then introduced to the cells via standard transfection or electroporation. 2) VirD2 and VirD1 are introduced in the host cell in constructs with inducible expression. The T-DNA is then introduced on a plasmid containing the border sequences via standard transformation. Processing of the T-strand then occurs in the cytoplasm upon induction of VirD1/VirD2 expression, before transport of the T-strand complex into the mitochondria. 3) Using an engineered Agrobacterium strain in which the native VirD2 sequence, encoded on a virulence plasmid, is modified to remove the NLS and introduce a MTS.

### Applications and Extendibility of Technologies to Other Systems

The demonstration of a CRISPR-Cas9 system, and its development as a tool is focused for the engineering of mitochondrial DNA of S. cerevisiae. It is shown how this system is capable of introducing mutations into the genome, and also how it can be used to insert fragments of DNA. A strategy is further laid down for how one can use screenable markers to increase the efficiency of the transformation process, and introduce methods for avoiding mitochondrial transformation with micro particle bombardment.

In yeast, engineering of medina may be used to improve the production of a class of chemicals which have industrial significance. The advantages of using this technology for bioproduction over nuclear integrations is the high copy number of the mitochondrial genome coupled with the abundant metabolites (acetyl-CoA, NADH) available for bioproduction in the mitochondrial matrix. More generally, the technologies described here should be applicable to mitochondrial genome editing in other eukaryotes. In human cells, mutations in mitochondrial encoded genes are responsible for a host of disease, and there is much interest in technologies to repair these through gene editing either in somatic cells (e.g., in the eye) or in cultured cells (e.g. stem cells) that can then be used as therapeutic agents. Finally, the strategies and technologies here can be broadly used for chloroplast genome editing in plants and algae, with the MTS sequences utilizes in each experiment substituted for plastid targeting sequences (PTS).

### REFERENCES CITED

1. Attardi, G. & Schatz, G. Biogenesis of mitochondria. Annu. Rev. Cell Biol. 4, 289-333 (1988).
2. Nunnari, J. & Suomalainen, A. Mitochondria: In Sickness and in Health. Cell 148, 1145-1159 (2017).
3. Danial, N. N. & Korsmeyer, S. J. Cell death: critical control points. Cell 116, 205-219 (2004).
4. Tait, S. W. G. & Green, D. R. Mitochondria and cell signalling. J. Cell Sci. 125, 807-815 (2012).
5. López-García, P. & Moreira, D. Open questions on the origin of eukaryotes. Trends Ecol. Evol. 30, 697-708 (2015).
6. Roger, A. J. Reconstructing Early Events in Eukaryotic Evolution. Am. Nat. 154, S146-S163 (1999).
7. Embley, T. M. & Martin, W. Eukaryotic evolution, changes and challenges. Nature 440, 623-630 (2006).
8. Koonin, E. V. Origin of eukaryotes from within archaea, archaeal eukaryome and bursts of gene gain: eukaryogenesis just made easier? Philos. Trans. R. Soc. B Biol. Sci. 370, (2015).
9. Gray, M. W. Mitochondrial evolution. Cold Spring Harb. Perspect. Biol. 4, a011403 (2012).
10. Yutin, N., Wolf, M. Y., Wolf, Y. I. & Koonin, E. V. The origins of phagocytosis and eukaryogenesis. Biol. Direct 4, 9 (2009).
11. Lane, N. & Martin, W. The energetics of genome complexity. Nature 467, 929-934 (2010).
12. Turk, E. M., Das, V., Seibert, R. D. & Andrulis, E. D. The Mitochondrial RNA Landscape of Saccharomyces cerevisiae. PLoS One 8, e78105 (2013).
13. Kucej, M. & Butow, R. A. Evolutionary tinkering with mitochondrial nucleoids. Trends Cell Biol. 17, 586-592 (2007).
14. Deshpande, A. P. & Patel, S. S. Interactions of the yeast mitochondrial RNA polymerase with the +1 and +2 promoter bases dictate transcription initiation efficiency. Nucleic Acids Res. 42, 11721-11732 (2014).
15. Lecrenier, N. & Foury, F. New features of mitochondrial DNA replication system in yeast and man. Gene 246, 37-48 (2000).
16. Savkina, M., Temiakov, D., McAllister, W. T. & Anikin, M. Multiple Functions of Yeast Mitochondrial Transcription Factor Mtf1p during Initiation. J. Biol. Chem. 285, 3957-3964 (2010).
17. Tang, G.-Q., Deshpande, A. P. & Patel, S. S. Transcription Factor-dependent DNA Bending Governs Promoter Recognition by the Mitochondrial RNA Polymerase. J. Biol. Chem. 286, 38805-38813 (2011).
18. Schatz, G. & Dobberstein, B. Common principles of protein translocation across membranes. Science 271, 1519-1526 (1996).
19. Wiedemann, N., Frazier, A. E. & Pfanner, N. The Protein Import Machinery of Mitochondria. J. Biol. Chem. 279, 14473-14476 (2004).
20. Hwang, W. Y. et al. Efficient In Vivo Genome Editing Using RNA-Guided Nucleases. Nat. Biotechnol. 31, 227-229 (2013).
21. DiCarlo, J. E. et al. Genome engineering in Saccharomyces cerevisiae using CRISPR-Cas systems. Nucleic Acids Res 41, (2013).
22. Wang, H. et al. One-Step Generation of Mice Carrying Mutations in Multiple Genes by CRISPR/Cas-Mediated Genome Engineering. Cell 153, 910-918 (2013).
23. Mali, P. et al. RNA-Guided Human Genome Engineering via Cas9. Science 339, 823-826 (2013).
24. Jinek, M. et al. A programmable dual-RNA-guided DNA endonuclease in adaptive bacterial immunity. Science 337, 816-821 (2012).
25. Jo, A. et al. Efficient Mitochondrial Genome Editing by CRISPR/Cas9. 2015, (2015).
26. Prasai, K., Robinson, L. C., Tatchell, K. & Harrison, L. Saccharomyces cerevisiae Mhr1 can bind Xho I-induced mitochondrial DNA double-strand breaks in vivo. Mitochondrion (2017). doi:https://doi.org/10.1016/j.mito.2017.10.005
27. Bacman, S. R., Williams, S. L. & Moraes, C. T. Intra- and inter-molecular recombination of mitochondrial DNA after in vivo induction of multiple double-strand breaks. Nucleic Acids Res. 37, 4218-4226 (2009).
28. Thyagarajan, B., Padua, R. A. & Campbell, C. Mammalian mitochondria possess homologous DNA recombination activity. J. Biol. Chem. 271, 27536-27543 (1996).
29. Kleff, S., Kemper, B. & Sternglanz, R. Identification and characterization of yeast mutants and the gene for a cruciform cutting endonuclease. EMBO J. 11, 699-704 (1992).
30. Kazak, L., Reyes, A. & Holt, I. J. Minimizing the damage: repair pathways keep mitochondrial DNA intact. Nat. Rev. Mol. Cell Biol. 13, 659 (2012).
31. Bonnefoy, N. & Fox, T. D. in (eds. Leister, D. & Herrmann, J. M.) 153-166 (Humana Press, 2007). doi:10.1007/978-1-59745-365-3_11
32. Larosa, V. & Remacle, C. Transformation of the mitochondrial genome. Int. J. Dev. Biol. 57, 659-665 (2013).
33. Gibson, D. G. et al. Enzymatic assembly of DNA molecules up to several hundred kilobases. Nat. Methods 6, 343 (2009).
34. Gietz, R. D. & Schiestl, R. H. High-efficiency yeast transformation using the LiAc/SS carrier DNA/PEG method. Nat. Protoc. 2, 31-34 (2007).
35. Gasiunas, G., Barrangou, R., Horvath, P. & Siksnys, V. Cas9-crRNA ribonucleoprotein complex mediates specific DNA cleavage for adaptive immunity in bacteria. Proc. Natl. Acad. Sci. U.S.A 109, E2579-86 (2012).
36. Sieber, F., Duchêne, A.-M. & Maréchal-Drouard, L. in (ed. Jeon, K. W. B. T.-I. R. of C. and M. B.) 287, 145-190 (Academic Press, 2011).
37. Entelis, N. et al. A glycolytic enzyme, enolase, is recruited as a cofactor of tRNA targeting toward mitochondria in Saccharomyces cerevisiae. Genes Dev. 20, 1609-1620 (2006).
38. Tarassov, I., Entelis, N. & Martin, R. P. An intact protein translocating machinery is required for mitochondrial import of a yeast cytoplasmic tRNA. J. Mol. Biol. 245, 315-323 (1995).
39. Herrmann, J. M. & Funes, S. Biogenesis of cytochrome oxidase-sophisticated assembly lines in the mitochondrial inner membrane. Gene 354, 43-52 (2005).
40. Paige, J. S., Wu, K. Y. & Jaffrey, S. R. RNA mimics of green fluorescent protein. Science 333, 642-646 (2011).
41. Thomas, D. Y. & Wilkie, D. Recombination of mitochondrial drug-resistance factors in Saccharomyces cerevisiae. Biochem. Biophys. Res. Commun. 30, 368-372 (1968).
42. Weisblum, B. Erythromycin resistance by ribosome modification. Antimicrob. Agents Chemother. 39, 577-585 (1995).
43. Sor, F. & Fukuhara, H. Erythromycin and spiramycin resistance mutations of yeast mitochondria: nature of the rib2 locus in the large ribosomal RNA gene. Nucleic Acids Res. 12, 8313-8318 (1984).
44. Sor, F. & Fukuhara, H. Identification of two erythromycin resistance mutations in the mitochondrial gene coding for the large ribosomal RNA in yeast. Nucleic Acids Res. 10, 6571-6577 (1982).
45. Scheiffele, P., Pansegrau, W. & Lanka, E. Initiation of Agrobacterium tumefaciens T-DNA Processing. Journal of Biological Chemistry 270, 1269-1276 (1995).
46. van Kregten, M., Lindhout, B. I., Hooykaas, P. J. J. & van der Zaal, B. J. Agrobacterium-mediated T-DNA transfer and integration by minimal VirD2 consisting of the relaxase domain and a type IV secretion system translocation signal. Mol. Plant. Microbe. Interact. 22, 1356-1365 (2009).
47. Jasper, F., Koncz, C., Schell, J. & Steinbiss, H. H. Agrobacterium T-strand production in vitro: sequence-specific cleavage and 5′ protection of single-stranded DNA templates by purified VirD2 protein. Proc. Natl. Acad. Sci. U.S.A 91, 694-698 (1994).
48. Vestweber, D. & Schatz, G. DNA-protein conjugates can enter mitochondria via the protein import pathway. Nature 338, 170-2 (1989).
49. Flierl, A. et al. Targeted delivery of DNA to the mitochondrial compartment via import sequence-conjugated peptide nucleic acid. Mol. Ther. 7, 550-557 (2003).
50. Ajikumar, P. K. et al. Terpenoids: Opportunities for Biosynthesis of Natural Product Drugs Using Engineered Microorganisms. Mol. Pharm. 5, 167-190 (2008).
51. Siddiqui, M. S., Thodey, K., Trenchard, I. & Smolke, C. D. Advancing secondary metabolite biosynthesis in yeast with synthetic biology tools. FEMS Yeast Res. 12, 144-170 (2012).
52. Nielsen, J. Synthetic Biology for Engineering Acetyl Coenzyme A Metabolism in Yeast. MBio 5, e02153-14 (2014).
53. Shiba, Y., Paradise, E. M., Kirby, J., Ro, D.-. K. & Keasling, J. D. Engineering of the pyruvate dehydrogenase bypass in Saccharomyces cerevisiae for high-level production of isoprenoids. Metab Eng 9, (2007).
54. Kozak, B. U. et al. Engineering acetyl coenzyme A supply: functional expression of a bacterial pyruvate dehydrogenase complex in the cytosol of Saccharomyces cerevisiae. MBio 5, e01696-14 (2014).
55. Farhi, M. et al. Harnessing yeast subcellular compartments for the production of plant terpenoids. Metab. Eng. 13, 474-481 (2011).

### Example 2

### Peptide-DNA Complexes that Deliver DNA into the Mitochondria

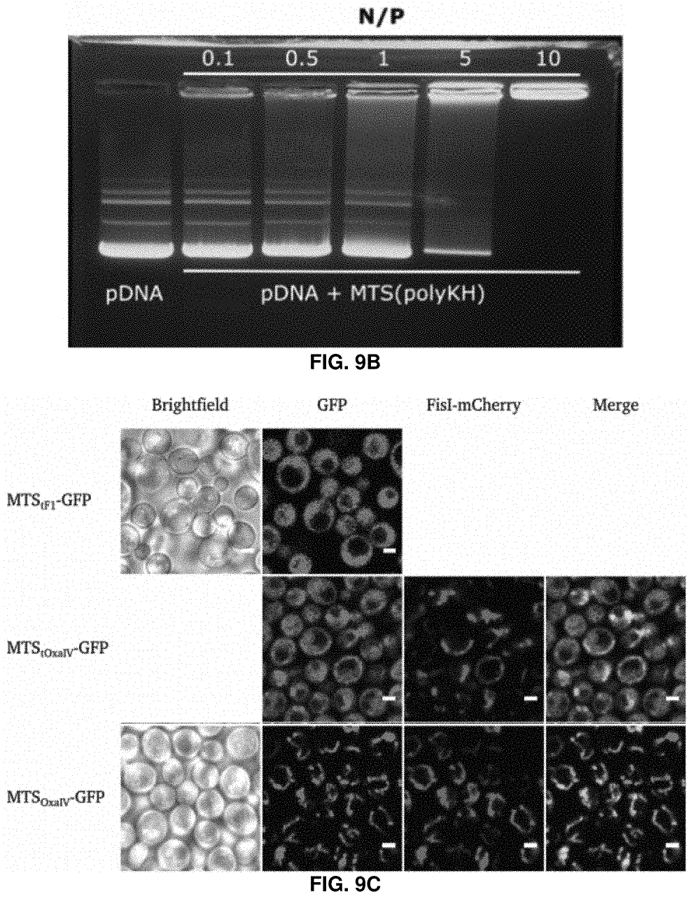

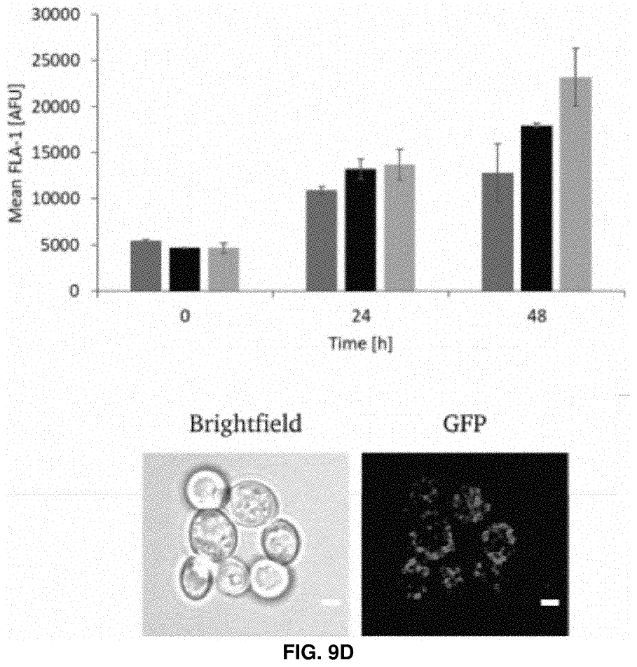

FIGS. 9A to 9D shows peptide-DNA complexes that deliver DNA into the mitochondria. A complex of two synthetic peptides (mitochondrial targeting peptide (MTP) and a cell penetrating peptide (CPP)) with the delivered DNA (pDNA) is transformed into cells, where it is targeted to the mitochondria where the pDNA is released. Peptide-DNA complex formation is assayed by electromobility retardation of pDNA with increasing amounts of peptide. A short (tOxaIV) mitochondrial targeting sequence (MTS) that targets GFP-tagged complexes to the mitochondria (imaged by the mitochondrial marker Fis1-mCherry) is designed. Demonstration of DNA delivery by transient expression of an mtDNA-encoded GFP gene off of the delivery pDNA. GFP is detected with increased bulk fluorescence (top) over the pDNA or peptide-free controls (green vs. black, red) and localized fluorescence in the mitochondria (bottom).

TABLE 1
Synthetic peptide sequences used in Example 2.

| Name | Sequence |
|---|---|
| MTP_{KH} | MLSLRQSIRFFKKHKHKHKHKHKHKHKHKH |
| | (SEQ ID NO: 1) |
| ACPP | KKLFKKILKYL |
| | (SEQ ID NO: 2) |
| YCPP | LLIILRRRIRKQAHAHSK |
| | (SEQ ID NO: 3) |

### Example 3

### Hijacking the Agrobacterium tumefaciens Virulence Machinery for T-DNA Targeting

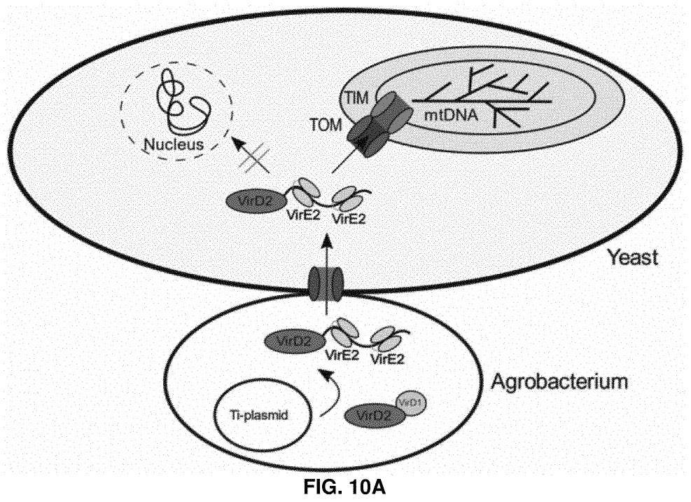

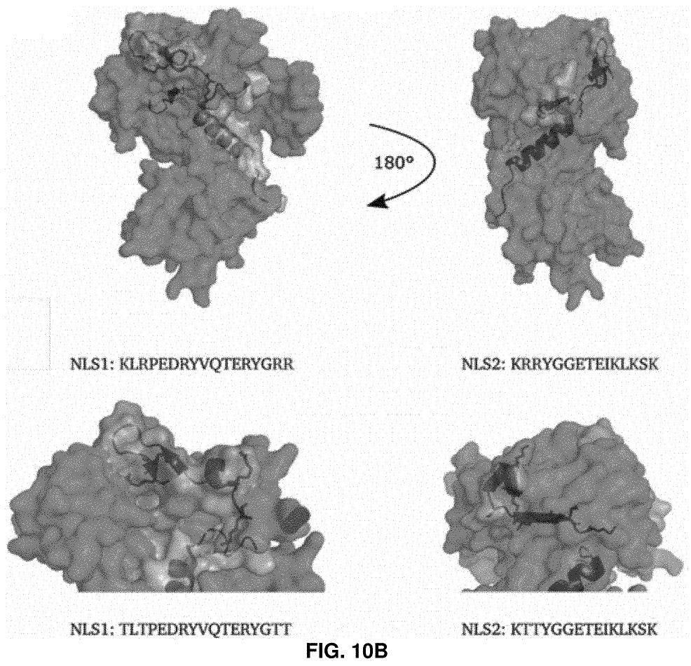

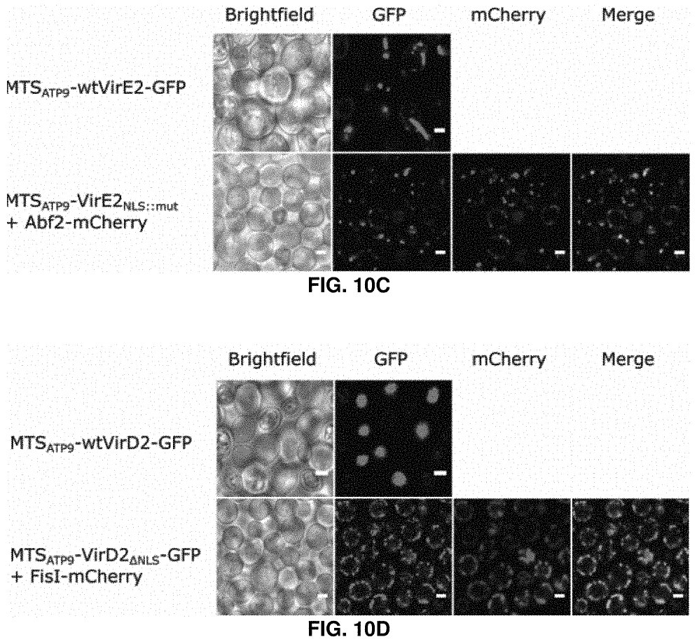

FIGS. 10A to 10D show the hijacking of the Agrobacterium tumefaciens virulence machinery for DNA targeting to mitochondria. This is an approach in which key proteins VirD2 and VirE2 are engineered so they not delivered to the nucleus but instead carry T-DNA encoded sequences to the mitochondria (mtDNA). Rational mutation of VirE2 nuclear localization sequences (NLS1 and NLS2) from their native, nucleus-targeted state to the engineered versions used here. Rational engineering of virulence (Vir) genes from Agrobacterium tumefaciens is used to target them away from the nucleus and into the mitochondria, which is confirmed by microscopy of fluorescent protein fusions to native VirE2 (top), which localizes to the nuclear chromosomes, and the engineered version (bottom), which co-localizes to mtDNA nucleoids labeled by Abf2-mCherry. Similar engineering of VirD2 relocalizes it from the nucleus to mitochondria. These proteins then deliver designer DNA into mitochondria provided that it is flanked by T-DNA recognition sequences native to the Vir system.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for editing a genome of a mitochondria or organelle, the method comprising: (a) introducing a targeted endonuclease, a targeting guide RNA, and a DNA fixing template into a mitochondria or organelle comprising a target DNA in the genome of the mitochondria or organelle, such that the DNA fixing template replaces the target DNA, (b) optionally selecting or screening for a phenotype of an eukaryotic cell comprising the mitochondria or organelle, wherein the DNA fixing template causes the phenotype.

2. A method for editing a genome of a mitochondria or organelle, the method comprising: (a) introducing a peptide-DNA complex comprising a peptide that targets a mitochondria complexed with a DNA fixing template into a mitochondria or organelle comprising a target DNA in the genome of the mitochondria or organelle, such that the DNA fixing template replaces the target DNA, and (b) optionally selecting or screening for a phenotype of an eukaryotic cell comprising the mitochondria or organelle, wherein the DNA fixing template causes the phenotype.

3. A method for editing a genome of a mitochondria or organelle, the method comprising: (a) providing one or more modified Agrobacterium tumefaciens Vir proteins wherein the one or more modified A. tumefaciens Vir proteins that target a DNA fixing template flanked by T-DNA recognition sequences native to the Vir system into a mitochondria or organelle instead of a nucleus, (b) introducing the one or more modified A. tumefaciens Vir proteins complexed to the DNA fixing template into an eukaryotic cell, (c) delivering the DNA fixing template into a mitochondria or organelle of the eukaryotic cell such that the DNA fixing template replaces a target DNA in the mitochondria or organelle, and (d) optionally selecting or screening for a phenotype of an eukaryotic cell comprising the mitochondria or organelle, wherein the DNA fixing template causes the phenotype.
